(19) United States
(12) Patent Application Publication
(10) Pub. No.: US 2023/0157906 A1
(43) Pub. Date: May 25, 2023

(54) ABSORBENT ARTICLE WITH AN EMBOSSED WAXED BARRIER

(71) Applicant: Kimberly-Clark Worldwide, Inc., Neenah, WI (US)

(72) Inventors: HyoungKun Park, YongIn-si (KR); JungHyun Park, YongIn-si (KR); Wonmi Jang, Seongnam-si (KR); David G. Biggs, New London, WI (US); YeonJung Kim, YongIn-si (KR); Jin Heo, SuWon-si (KR); HwaJun Lee, YongIn-si (KR); SangMin Lee, SeongNam-si (KR); JuHyung Lee, SuWon-si (KR); SeongDae Roh, Neenah, WI (US); HyungWoo Park, SuWon-si (KR); HyeJin Lee, Seoul-si (KR)

(21) Appl. No.: 16/971,241

(22) PCT Filed: Feb. 28, 2018
(86) PCT No.: PCT/US18/20337

§ 371 Date: Aug. 19, 2020

(51) Int. Cl. A61F 13/511; A61L 15/22; A61F 13/513; A61F 13/15

(52) U.S. Cl. CPC A61F 13/51104; A61L 15/225; A61F 13/51394; A61F 13/15731

(57) ABSTRACT

The present disclosure presents a stain reduction improvement of a disposable absorbent article by applying a hydrophobic barrier formulation to a topsheet and/or absorbent core area of the article and thereafter embossing the treated area with heat and pressure. A barrier is thus created wherein the barrier is along the full length of the embossment.

### TECHNICAL FIELD

The present disclosure relates to embossing an absorbent article such as a sanitary napkin or an incontinent pad. In particular, it is directed to an apparatus and methods of applying a hydrophobic barrier formulation onto a disposable absorbent article and embossing the barrier formulation by heat and pressure thereby creating a barrier along the full length of the embossed area which may extend from the top of a topsheet through an absorbent core to the top of a backsheet.

### BACKGROUND OF THE DISCLOSURE

Disposable absorbent articles, such as, pantiliners, sanitary napkins, interlabial devices, adult incontinence devices, bandages, wipes, diapers and the like are well known. These articles typically have a fluid permeable body-facing side and fluid impermeable garment facing side. Additionally, such articles may include an absorbent layer for retaining fluids there between.

Absorbent articles having patterns imposed upon their surfaces are on the market. For example, sanitary protection articles, such as, napkins, pantiliners, and incontinence devices, typically have a cover of nonwoven material having a pattern of depressed areas embossed into the surface in such configurations as flowers or other feminine designs. Other patterns may take the form of various geometric shapes, such as, circles, diamonds, squares, curves, or other stylized figures, such as, stars, dots, or the like.

While articles are embossed mainly for decorative purposes, embossing may communicate or provide a function to the user of such an article. For example, it is known that embossing functions in impeding or directing fluid flow.

Embossing methods for absorbent articles are well known. For example, U.S. Pat. No. 4,518,451 discloses an embossed pantiliner where the body facing side of the absorbent body has imposed therein a pattern of relatively deep depressed areas whereas the longitudinal edges have imposed therein a pattern of relatively shallow depressed areas. This results in a pantiliner that has aesthetically effective embossing while providing comfortable longitudinal edges. U.S. Pat. No. 4,623,340 discloses an absorbent pantiliner provided with a pattern of depressed areas. The outer cover of the pantiliner is made from a relatively light opaque sheet material and the interior layer is at least partially thermoplastic and relatively dark colored. When the cover is embossed, the depressed areas appear darker than the unembossed areas. Embossing is also done to provide a visual cue to show differences in the material surface. Embossing, however, has yet to be used to improve leakage protection (or reduce staining) in a disposable absorbent article when used at a specified temperature and pressure when embossing specific barrier formulations.

The present disclosure presents a stain reduction improvement of a disposable absorbent article by utilizing a hydrophobic barrier formulation and embossing it on the topsheet and/or through the absorbent core to the top of the backsheet to create a barrier wherein the barrier is formed along the full length of the embossment.

### SUMMARY OF THE DISCLOSURE

The present disclosure provides for a disposable absorbent article having a liquid permeable topsheet, a liquid impermeable backsheet, and an absorbent core interposed between the topsheet and backsheet, wherein a hydrophobic barrier formulation is embossed by heat and pressure onto the absorbent article so as to form a barrier wherein a barrier is formed along the full length of the embossment.

The absorbent article of the present disclosure surprisingly and unexpectedly prevents side leakage by embossing a barrier formulation with heat and pressure so as to suppress the surface flow of liquid and the oozing of liquid in the topsheet or absorbent core which cause side leakage.

Most absorbent article barrier formulations are hydrophobic anhydrous formulations that consist of base ingredients, waxes, rheology modifier, and additives. In one embodiment the current disclosure uses simply bi-component with hydrophobic and economical ingredients such as petrolatum and ozokerite wax.

In one embodiment a disposable absorbent article may comprise:
a) an outer cover;
b) a liquid permeable topsheet which is connected in superposed relation to the outer cover;
c) an absorbent core which is located between the topsheet and a liquid impermeable backsheet;
d) a barrier formulation on at least a portion of the topsheet surface which includes from about 5 to about 100 weight percent of a base ingredient, from about 0 to about 95 weight percent of a wax, and from about 0 to about 25 weight percent of a rheology modifier
   alternatively, in another embodiment of the disclosure, the barrier formulation may be placed on the absorbent core as opposed to the topsheet surface. The barrier formulation includes from about 5 to about 100 weight percent of a base ingredient, from about 0 to about 95 weight percent of a wax, and from about 0 to about 25 weight percent of a rheology modifier; and
e) an embossment pattern wherein the embossment pattern is applied onto the barrier treated area whereby forming a barrier along the full length of the embossment.
   In a further embodiment, according to the preceding embodiment, a second barrier formulation may be applied wherein a second barrier is formed on top of the absorbent article.

In another embodiment, the disposable absorbent article, according to the preceding embodiments, may be pantiliners, sanitary napkins, interlabial devices, adult incontinence devices, bandages, wipes, diapers and the like.

In yet another embodiment, according to the preceding embodiments, wherein said base ingredient is selected from the group consisting of petroleum based oils, vegetable oils, mineral oils, natural or synthetic oils, silicone oils, lanolin and lanolin derivatives, (esters) eucalyptol, cetearyl glucoside, dimethyl isosorbicide, polyglyceryl-3 cetyl ether, polyglyceryl-3 decyltetradecanol, propylene glycol myristyl ether, alkoxylated carboxylic acid, alkoxylated alcohols, (fatty alcohols) octyldodecanol, lauryl alcohol, myristyl alcohol, cetyl alcohol, stearyl alcohol, cetearyl alcohol, behenyl alcohol and mixtures thereof.

In a further embodiment, according to the preceding embodiments, the base ingredient is a petroleum based ingredient.

In another embodiment, according to the preceding embodiments, wherein the wax is selected from the group consisting of animal wax, vegetable wax, mineral wax, silicone wax, natural or synthetic wax, vbayberry wax, bees wax, C30 alkyl dimethicone, candelila wax, carnauba wax, ceresin wax, cetyl esters, esparto, hydrogenated cotton seed oil, hydrogenated jojoba oil, hydrogenated jojoba wax, hydrogenated microcrystalline wax, hydrogenated rice bran wax, japan wax, jojoba wax, jojoba butter, jojoba esters, lanolin wax, microcrystalline wax, mink wax, motan acid wax, motan wax, ouricury wax, ozokerite wax, paraffin wax, PEG-6 beeswax, PEG-8 beeswax, rice bran wax, shellac wax, spent grain wax, spermaceti wax, stearyl dimethicone, synthetic beeswax, synthetic candelilla wax, synthetic jojoba wax and mixtures thereof all of which may be natural or synthetic.

In a further embodiment, accordingly to the preceding embodiments, the rheology modifier is polyolefin resins, lipophilic/oil thickeners, ethylene/vinyl acetate copolymers, polyethylene, silica, talc, colloidal silicone dioxide, zinc stearate, cetyl hydroxyl ethyl cellulose, or a modified cellulose.

In another embodiment, accordingly to the preceding embodiments, the barrier formulation is a combination of base ingredients and waxes.

In a further embodiment, accordingly to the preceding embodiments, the barrier formulation is about 35-45% ozokerite wax and about 55% to about 65% petrolatum.

In yet another embodiment, according to the preceding embodiments, the barrier formulation is about 35-45% stearyl alcohol and about 55% to about 65% petrolatum.

In still another embodiment, according to the preceding embodiments, the barrier formulation is from about 55%-65% castrolatum and from about 35-45% cetearyl alcohol.

In yet a further embodiment, according to the preceding embodiments, the barrier formulation is from about 55-65% Sebapet L and from about 35-45% stearyl alcohol.

In an additional embodiment, according to the preceding embodiments, the barrier formulation is from about 55-65% Sonnenatural J-207 and from about 35-45% stearyl alcohol.

In a further embodiment, according to the preceding embodiments, the barrier formulation is about 2-5% bees wax, about 15-20% stearyl alcohol, about 15-20% behenyl alcohol, about 25-30% cetyl palmitate and about 1-4% glyceryl oleate.

In yet another embodiment, according to the preceding embodiments, the barrier formulation is about 30-34% bees wax, about 15-20% stearyl alcohol, about 15-20% behenyl alcohol, and about 25-30% cetyl palmitate.

In an additional embodiment, according to the preceding embodiments, the barrier formulation is about 15-18% bees wax, about 52-56% cetyl palmitate and about 26-30% glyceryl oleate.

In yet another embodiment, according to the preceding embodiments, the barrier formulation is applied to the top sheet at a temperature no more than about 10 degrees centigrade above a melting point of the barrier formulation to reduce migration of the barrier formulation.

In another embodiment, according to the preceding embodiments, the barrier formulation is applied to the absorbent core at a temperature no more than about 10 degrees centigrade above a melting point of the barrier formulation to reduce migration of the barrier formulation.

In a further embodiment, according to the preceding embodiments, wherein the barrier formulation is applied to the top sheet with a pressure of about 1.0 kgf/cm2 to about 7.5 kgf/cm2.

In yet another embodiment, according to the preceding embodiments, wherein the barrier formulation is applied to the absorbent core with a pressure of about 1.0 kgf/cm2 to about 7.5 kgf/cm2.

In another embodiment, according to the preceding embodiments, wherein the embossed pattern may be a continuous pattern such as a line or may be in a discontinuous pattern such as geometric shapes like rectangles, diamonds, squares, circles, letters, numbers, images, or combinations thereof.

In a further embodiment, according to the preceding embodiments, wherein the embossed pattern may be any shape, like a cone, cube, rectangle, rounded, oval, hatch marks, criss-crosses, bars, waves, spirals, swirls, letters, words, numbers, logos, geometric shapes, and combinations thereof.

In yet another embodiment, according to the preceding embodiments, wherein the barrier is invisible.

In still another embodiment, according to the preceding embodiments, wherein the barrier extends from beneath the top sheet to the back sheet.

In yet another embodiment, according to the preceding embodiments, wherein the barrier extends from the top sheet to the inside of the absorbent core.

In an additional embodiment, according to the preceding embodiments, wherein the barrier formulation is hydrophobic.

In yet a further embodiment, according to the preceding embodiments, wherein the embossment has an embossing pattern having a plurality of embossments.

In yet an additional embodiment, according to the preceding embodiments, wherein shape of the top of the top sheet is selected from the group consisting of flat, curved, pointed, irregular planes, and combinations thereof to produce an embossed area.

In a further embodiment, according to the preceding embodiments, wherein embossing pattern between the anterior article (or dimple) pattern and the treated embossed area is at least 3 mm apart from each other.

In an additional embodiment, according to the preceding embodiments, wherein the embossing pattern thickness is at least 15% of the total thickness of the absorbent article. More particularly, the embossing pattern thickness may be at least 4 mm.

In yet an additional embodiment, according to the preceding embodiments, wherein the embossing pattern has a preferable width from about 9 mm to about 17 mm and the length of the pattern is from about 70 mm to about 100 mm. If the side embossing pattern was enlarged then the length of the absorbent article would also be lengthened.

In another embodiment, a process for making a barrier in a disposable absorbent article comprises:
applying a barrier formulation onto the top surface of a body facing topsheet;
embossing the barrier formulation with heat and pressure;
whereby forming a barrier along the full length of the embossed area thus creating a barrier that extends from the top sheet through an absorbent core to a back sheet.

In yet another embodiment, according to the preceding embodiment, wherein the disposable absorbent article may be pantiliners, sanitary napkins, interlabial devices, adult incontinence devices, bandages, wipes, diapers and the like.

In a further embodiment, according to the preceding embodiment, another second barrier formulation may be applied wherein a second barrier is formed on top of the absorbent article.

In another embodiment, according to the preceding embodiments, wherein the barrier formulation is a combination of base ingredients and waxes.

In yet another embodiment, according to the preceding embodiments, wherein the barrier formulation is about 35-45% ozokerite wax and about 55% to about 65% petrolatum.

In another embodiment, according to the preceding embodiments, wherein the barrier formulation is about 35-45% stearyl alcohol and about 55% to about 65% petrolatum.

In still another embodiment, according to the preceding embodiments, wherein the barrier formulation is from about 55%-65% castrolatum and from about 35-45% cetearyl alcohol.

In yet a further embodiment, according to the preceding embodiments, the barrier formulation is from about 55-65% Sebapet L and from about 35-45% stearyl alcohol.

In an additional embodiment, according to the preceding embodiments, the barrier formulation is from about 55-65% Sonnenatural J-207 and from about 35-45% stearyl alcohol.

In yet another embodiment, according to the preceding embodiments, wherein the barrier formulation is about 2-5% bees wax, about 15-20% stearyl alcohol, about 15-20% behenyl alcohol, about 25-30% cetyl palmitate and about 1-4% glyceryl oleate.

In yet an additional embodiment, according to the preceding embodiments, wherein the barrier formulation is about 30-34% bees wax, about 15-20% stearyl alcohol, about 15-20% behenyl alcohol, and about 25-30% cetyl palmitate.

In yet another embodiment, according to the preceding embodiments, wherein the barrier formulation is about 15-18% bees wax, about 52-56% cetyl palmitate and about 26-30% glyceryl oleate.

In yet a further embodiment, according to the preceding embodiments, wherein the barrier formulation is applied to the top sheet at a temperature no more than about 10 degrees centigrade above a melting point of the barrier formulation to reduce migration of the barrier formulation.

In yet an additional embodiment, according to the preceding embodiments, wherein the barrier formulation is applied to the top sheet with a pressure of about 1.0 kgf/cm2 to about 7.5 kgf/cm2.

In yet a further embodiment, according to the preceding embodiments, wherein the barrier formulation is applied to the absorbent core with a pressure of about 1.0 kgf/cm2 to about 7.5 kgf/cm2.

In yet another embodiment, according to the preceding embodiments, wherein the embossed area may be a continuous pattern such as a line or may be in a discontinuous pattern such as geometric shapes like rectangles, diamonds, squares, circles, letters, numbers, images, or combinations thereof.

In an additional embodiment, according to the preceding embodiments, wherein the embossed area may be any shape, like a cone, cube, rectangle, rounded, oval, hatch marks, criss-crosses, bars, waves, spirals, swirls, letters, words, numbers, logos, geometric shapes, and combinations thereof.

In another embodiment, according to the preceding embodiments, wherein the barrier is invisible.

In yet another embodiment, according to the preceding embodiments, wherein the barrier extends from beneath the top sheet to the back sheet.

In yet a further embodiment, according to the preceding embodiments, wherein the barrier extends from the top sheet to the inside of the absorbent core.

In another embodiment, according to the preceding embodiments, wherein the barrier formulation is hydrophobic.

In yet another embodiment, according to the preceding embodiments, wherein the embossed area comprises an embossing pattern having a plurality of embossments.

In still another embodiment, according to the preceding embodiments, wherein the shape of the top of the top sheet is selected from the group consisting of flat, curved, pointed, irregular planes, and combinations thereof to produce an embossed area.

In yet a further embodiment, according to the preceding embodiments, wherein the embossing pattern thickness is at least 15% of the total thickness of the absorbent article. More particularly the embossed pattern may be at least 4 mm.

In a further embodiment, according to the preceding embodiments, wherein embossing pattern between the anterior article (or dimple) pattern and the treated embossed area is at least 3 mm apart from each other.

In yet an additional embodiment, according to the preceding embodiments, wherein the embossing pattern has a preferable width from about 9 mm to about 17 mm and the length of the pattern is from about 70 mm to about 100 mm. If the side embossing pattern was enlarged then the length of the absorbent article would also be lengthened.

In yet an additional embodiment, a process for making a barrier in a disposable absorbent article comprises:
applying a barrier formulation onto an absorbent core;
embossing the barrier formulation with heat and pressure;
whereby forming a barrier along the full length of the embossed area thus creating a barrier that extends from the absorbent core to a back sheet.

In a further embodiment, according to the preceding embodiment, another second barrier formulation may be applied wherein a second barrier is formed on top of the absorbent article.

In yet another embodiment, according to the preceding embodiment, wherein the disposable absorbent article may be pantiliners, sanitary napkins, interlabial devices, adult incontinence devices, bandages, wipes, diapers and the like.

In another embodiment, according to the preceding embodiments, wherein the barrier formulation is a combination of base ingredients and waxes.

In yet another embodiment, according to the preceding embodiments, wherein the barrier formulation is about 35-45% ozokerite wax and about 55% to about 65% petrolatum.

In a further embodiment, according to the preceding embodiments, wherein the barrier formulation is about 35-45% stearyl alcohol and about 55% to about 65% petrolatum.

In still another embodiment, according to the preceding embodiments, the barrier formulation is from about 55%-65% castrolatum and from about 35-45% cetearyl alcohol.

In yet a further embodiment, according to the preceding embodiments, the barrier formulation is from about 55-65% Sebapet L and from about 35-45% stearyl alcohol.

In an additional embodiment, according to the preceding embodiments, the barrier formulation is from about 55-65% Sonnenatural J-207 and from about 35-45% stearyl alcohol.

In yet another embodiment, according to the preceding embodiments, wherein the barrier formulation is about 2-5% bees wax, about 15-20% stearyl alcohol, about 15-20% behenyl alcohol, about 25-30% cetyl palmitate and about 1-4% glyceryl oleate.

In yet a further embodiment, according to the preceding embodiments, wherein the barrier formulation is about 30-34% bees wax, about 15-20% stearyl alcohol, about 15-20% behenyl alcohol, and about 25-30% cetyl palmitate.

In yet another embodiment, according to the preceding embodiments, wherein the barrier formulation is about 15-18% bees wax, about 52-56% cetyl palmitate and about 26-30% glyceryl oleate.

In yet a further embodiment, according to the preceding embodiments, wherein the barrier formulation is applied to the top sheet at a temperature no more than about 10 degrees centigrade above a melting point of the barrier formulation to reduce migration of the barrier formulation.

In yet another embodiment, according to the preceding embodiments, wherein the barrier formulation is applied to the top sheet with a pressure of about 1.0 kgf/cm2 to about 7.5 kgf/cm2.

In yet another embodiment, according to the preceding embodiments, wherein the barrier formulation is applied to the absorbent core with a pressure of about 1.0 kgf/cm2 to about 7.5 kgf/cm2.

In yet an additional embodiment, according to the preceding embodiments, wherein the embossed area may be a continuous pattern such as a line or may be in a discontinuous pattern such as geometric shapes like rectangles, diamonds, squares, circles, letters, numbers, images, or combinations thereof.

In yet another embodiment, according to the preceding embodiments, wherein the embossed area may be any shape, like a cone, cube, rectangle, rounded, oval, hatch marks, criss-crosses, bars, waves, spirals, swirls, letters, words, numbers, logos, geometric shapes, and combinations thereof.

In yet a further embodiment, according to the preceding embodiments, wherein the barrier is invisible.

In yet an additional embodiment, according to the preceding embodiments, wherein the barrier extends from beneath the absorbent core to the back sheet.

In yet a further embodiment, according to the preceding embodiments, wherein the barrier formulation is hydrophobic.

In yet another embodiment, according to the preceding embodiments, wherein the embossed area comprises an embossing pattern having a plurality of embossments.

In a further embodiment, according to the preceding embodiments, wherein embossing pattern between the anterior article (or dimple) pattern and the treated embossed area is at least 3 mm apart from each other.

In yet a further embodiment, according to the preceding embodiments, wherein the embossing pattern thickness is at least 15% of the total thickness of the absorbent article. More particularly the embossed pattern may be at least 4 mm.

In yet an additional embodiment, according to the preceding embodiments, wherein the embossing pattern has a preferable width from about 9 mm to about 17 mm and the length of the pattern is from about 70 mm to about 100 mm. If the side embossing pattern was enlarged then the length of the absorbent article would also be lengthened.

The above summary of the present disclosure is not intended to describe each embodiment or every implementation of the present disclosure. Advantages and attainments, together with a more complete understanding of the disclosure, will become apparent and appreciated by referring to the following detailed description and claims taken in conjunction with the accompanying drawings.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### BRIEF DESCRIPTION OF DRAWINGS

The disclosure may be more completely understood in consideration of the following detailed description of various embodiments in connection with the accompanying drawing, in which:

FIG. 1 shows a disposable absorbent article where one side is treated with embossing patterns that are applied on a barrier treated area versus an untreated side;

FIG. 2 shows various disposable absorbent articles where one side is treated with embossing patterns that are applied on a barrier treated area versus an untreated side.

While the disclosure is amenable to various modifications and alternative forms, specifics thereof have been shown by way of example in the drawing and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION OF THE DISCLOSURE

Each example of this disclosure is provided by way of explanation and is not meant as a limitation. For example, features illustrated or described as part of one embodiment or figure can be used on another embodiment or figure to yield yet another embodiment. It is intended that the present disclosure include such modifications and variations.

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

When introducing elements of the present disclosure or the preferred embodiment(s) thereof, the articles “a”, “an”, and “the” are intended to mean that there are one or more of the elements.

The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

The current disclosure presents the application of a barrier formulation onto design patterns on a BSL (Body Side Liner) of a disposable absorbent article. The design patterns on the liner are thereafter embossed. Heat and pressure during the embossing process melted the barrier. The molten barrier penetrated into the article layers and built hydrophobic barriers after cooling down.

A barrier formulation may be an ointment or any formulation where a barrier is formed to inhibit or stop a liquid from seeping through an absorbent article.

A rheology modifier may be a viscosity enhancer or any similar type of tool that may aid in enhancing the thickness.

Disposable absorbent articles typically include a cover wherein a body facing topsheet is enclosed and a backsheet or a garment facing layer. Additionally, an absorbent core, transfer layer, construction and positioning adhesive, and release paper may be included in the absorbent article construction.

Generally, a barrier formulation is applied to a disposable absorbent article wherein the formulation changes from a liquid to a solid and thereafter is embossed to a liquid by heat and pressure disclosed herein and thereafter changed back to a solid thus forming a barrier on or in the absorbent article.

Cover and Body-Facing Topsheet

The cover of an absorbent article includes the body-facing topsheet. In one embodiment, the cover may be a layer of material that is juxtaposed on the absorbent core.

As known by those skilled in the art, the cover may be formed from any fluid pervious material that is comfortable against the skin and permits fluid to penetrate to the absorbent core, which retains the fluid. The cover should retain little or no fluid to provide a relatively dry surface next to the skin when in use. The cover can be a single sheet or layer of material having a width sufficient to form the body-facing surface of the absorbent article.

A variety of cover materials are known in the art, and any of these may be used. For instance, the cover has been made from fibrous non-woven fabrics made of fibers or filaments of polymers, such as polyethylene, polypropylene, polyester, or cellulose, and combinations or mixtures thereof. The fiber or filament can be single denier or multidenier.

Other materials used in making covers include gauze or for example, a nonwoven material such as the ones described in U.S. Pat. No. 3,554,788 (Fechillas), any known porous material with a suitable body contacting surface, including, but not limited to nonwoven webs, apertured films, plastic nets, and the like. Previously, the cover could also be made from a fibrous nonwoven composite of bicomponent fibers and pulp fluff.

Alternatively, the cover may be formed from an apertured polymeric film. In addition, such a film may be treated with a surfactant to increase hydrophilicity.

Additionally, the material used to form the cover should be hydrophilic, which can be obtained by any methods known to those skilled in the art, such as surface treatment, surface spraying, or incorporation of a surfactant into the material.

Transfer Layer

The absorbent article of the present invention may have an optional transfer layer. The transfer layer may be made of any known material that will take up fluid and then distribute and release it to an adjacent absorbent core or layer for storage. Transfer layers often have a relatively open structure that allows for movement of fluid within the layer. Suitable materials for such transfer layers include fibrous webs, resilient foams, and the like.

The transfer layer is able to accept fluid and allow passage of the fluid through its mass to be absorbed by an adjacent absorbent core. Thus, transfer layers that are made of hydrophobic, nonabsorbent fibers may be able to accept large volumes of fluid into interfiber void spaces while the fibers themselves do not absorb any significant quantities of fluid. Likewise, open-celled foam structures that are made from nonabsorbent materials may also absorb fluid into the cells of the foam. The walls of the cells, however, do not absorb any fluid. The cumulative spaces within the transfer layer, i.e., the interfiber void spaces in the fibrous transfer layer or the open cells in the foam transfer layer, function much like a container to hold fluid.

Transfer layers that are made from webs of mostly absorbent fibers absorb the fluid as it enters the structure and do not distribute it throughout the rest of the structure as efficiently as webs containing non-absorbent materials. Preferred transfer layer fibrous webs include nonabsorbent materials to provide void volume and to allow for free movement of fluid through the structure. Examples of preferred materials include polypropylene, polyethylene, polyester, bicomponent materials, nylon and mixtures or combinations thereof.

The transfer layer does not have to be apertured film; it can be any other nonwoven material, such as, foam or netting, which transports fluids.

The cover layer can be joined or laminated to the transfer layer by any methods known in the art such as fusion bonding, adhesive attachment, or by any other methods for securing layers together. Fusion bonding includes heat bonding, ultrasonic bonding, and the like.

In another embodiment, the cover and transfer layers are joined together by first applying adhesive to the underside of the cover material and placing the cover web onto the transfer layer material. While any adhesive may be used, such as any non-pressure sensitive adhesive, a preferred adhesive is D 1280 BE (available from Fuller Co., Germany).

In one embodiment, the composite layer formed from the cover and transfer layers may be further processed.

Absorbent Core

The absorbent core or layer of the present invention may contain any known absorbent materials including, but not limited to, absorbent fibers, such as cellulose fibers, including, but not limited to wood pulp, regenerated cellulose fibers, and cotton fibers, rayon fibers and the like; superabsorbent fibers or particles; other naturally occurring absorbent materials, such as peat moss; and other synthetic absorbent materials, such as foams and the like. The absorbent layer may also include one or more of the following: thermoplastic binder fibers, latex binder, perfumes, oils or odor-controlling compounds. Additionally, the absorbent layer may be a mixture of two or more types of thermoplastic fibers having different melting points. Bicomponent fibers, fibers with an inner core of a thermoplastic fiber, e.g., polyester, surrounded by an outer sheath of thermoplastic, e.g., polyethylene, having a melting point much lower than the core, have been found to be the best fibers to work with from processing and performance standpoints. Upon application of heat and pressure sufficient to melt at least one of the fiber types, the remaining unmelted fibers will be thermobonded or fused together into a porous web.

Cellulosic pulp fibers can also be included with thermoplastic fibers. Since thermoplastic fibers, without further treatment, are essentially hydrophobic, the absorbent layer will not effectively draw fluid away from the composite cover and transfer layer absent some hydrophilic material. It is important to have sufficient pulp to absorb fluid.

In a particularly useful embodiment, the absorbent layer is made from airlaid pulp and includes about 15 percent by weight of superabsorbent polymer available as Salsorb CL 15 from Chemdal (United Kingdom). The amount of superabsorbent in the absorbent core may be in the range of from about 5 to about 50 percent by weight of the absorbent core. The absorbent core may be compressed or uncompressed, embossed, or calendered.

Backsheet

The backsheet of the present disclosure is a body fluid impervious material, typically referred to as a “barrier,” which is at least substantially impermeable to liquids. Its exterior forms the garment-facing surface of the absorbent article. The backsheet may be any thin, flexible, body fluid impermeable material, such as, but not limited to, a polymeric film, e.g., polyethylene, polypropylene, or cellophane, or a normally fluid pervious material that has been treated to be impervious, such as impregnated fluid repellent paper or non-woven material, including non-woven fabric material, or a flexible foam, such as polyurethane or cross-linked polyethylene. In the present invention, the backsheet may be a mixture of material and pigment. The thickness of the backsheet may be from about 0.0005 to about 0.002 inch (about 0.013 mm to about 0.051 mm).

Optionally, the backsheet may be breathable, i.e., permits vapor to transpire. Known materials for this purpose include nonwoven materials, monolithic and microporous films in which microporosity is created by, inter alia, stretching an oriented film. Single or multiple layers of permeable films, fabrics, melt-blown materials, and combinations thereof that provide a tortuous path, and/or whose surface characteristics provide a liquid surface repellent to the penetration of liquids may also be used to provide a breathable backsheet.

Absorbent Structure

An absorbent structure is formed from an absorbent core and a film, which have been laminated together to form a unitary structure. The film may be any water insoluble, biodegradable polymers, such as those used as the cover or alternately the film, may be made from materials, such as, PCL, PLA, and PVOH.

The film may be adhered to the absorbent core by any method known to one of skill in the art. Such methods of adherence include mechanical methods, such as, heating, embossing, crimping, hooks, and the like, and chemical methods, such as, adhesives, including latex, solvents, and the like.

In one embodiment, an absorbent structure can be made of an absorbent core made of a wet laid pulp, available as Valucore brand from Rayonier, Jessup, Ga., which is laminated using hot melt adhesive to a 1 mil layer of PCL (monomer available from Union Carbide/Dow, Midland, Mich.).

An outer shell (not shown) is water dispersible and may be made of any water soluble, fluid impermeable material. It may be formed from a laminate, composite or blend. It can also be a blend of water soluble and water insoluble materials or a composite of water soluble and water insoluble material. By having the water soluble component in the blend or composite, the water insoluble component will break apart upon exposure to water and agitation.

In one embodiment, a layer of water insoluble, biodegradable material is laminated or coated with a water soluble material. For example, an outer shell can be formed by coating a water-soluble PVOH resin to paper (cellulose fibers) on one side to form a laminated structure. The paper itself is not water soluble but can be biodegraded. PVOH is soluble upon exposure to water.

In another embodiment, a water insoluble material is blended with a water soluble material that acts as a binder. For example, a polyester material may be blended with a water soluble material, such as, PVOH. An example of this embodiment are blends of PCL and PVOH including about 25:about 75, about 50:about 50, and about 75:about 25 weight/weight blends that were melt extruded at about 350 degrees to about 420 degrees F. (about 177 to about 216 degrees C.) at about 10 RPM into films.

Other examples of water insoluble, biodegradable materials include polyortho esters, polyanhydrides, tyrosine-based polymers, polyphosphoesters, polyetheramides, polyesters including, but not limited to, poly(butylene succinate), poly(butylene succinate-co-adipate), poly(butylene succinate-co-carbonate), poly(butylene succinate-co-terephthalate), poly(ethylene succinate-co-adipate), poly(butylene adipate-co-terephthalate), poly(tetramethylene adipate-co-terephthalate), PCL, PLA, and bacterial polyesters, such as, poly(hydroxyalkanoates). Natural polymers, such as, polysaccharides including starch and their blends, cellulose and their derivatives and biosynthetic polysaccharides may also be used.

Different types of water dispersable papers may also be used. In one embodiment, the outer shell may have two layers: a layer of water soluble paper and a layer of PVOH. In another embodiment, the outer shell may have three layers: a layer of water soluble paper coated on both sides by PVOH. Alternately, the paper pulp may be mixed with a water soluble material. For example, the water soluble paper components may be mixed with PVOH prior to forming the layer of water soluble paper. The PVOH may act as a binder and be used to increase the strength and integrity of the paper layer. For example, Mishima Paper Co., LTD manufactures water soluble paper made from cellulose and CMC that can dissolve in water in about 10 to about 180 seconds. In particular, types A3015 and B3015 are extrusion laminated with PVOH and may be heat sealed to the absorbent structure. Other types of paper may be attached to the absorbent using construction adhesive or attached by other means. In another embodiment, A3015 paper (Mishima Paper Co., LTD) is laminated to the absorbent structure.

Bonding

The layers of the absorbent article may be, but not necessarily, bonded, e.g., glued or adhered, to an adjacent layer. For example, the underside of the cover layer is adhered to the top side of the absorbent layer. The underside of the absorbent layer is adhered to the top side of the backsheet. As previously stated, any methods known in the art, such as fusion bonding, adhesive attachment, or by any other securement means can be used to bond the individual layers together to form the final absorbent article. Fusion bonding includes heat bonding, ultrasonic bonding, and the like.

Any construction adhesive may be used to attach the layers into a single absorbent article. For example, in one embodiment, the body facing layer is attached to the outer shell with adhesive HL 1491 available from H.B Fuller and Company (St. Paul, Minn.). The adhesive may be applied by any method known to those skilled in the art.

The absorbent article may be applied to the crotch of a garment by placing the garment-facing surface against the inside surface of the crotch of the garment. Pressure sensitive adhesive may be applied to the garment-facing surface of the absorbent article to help secure it in place. As used herein, the term “pressure-sensitive adhesive” refers to any releasable adhesive or releasable tenacious means. Suitable pressure sensitive adhesives include for example water-based adhesives such as acrylate adhesives. Alternatively, the adhesive may comprise “hot melt” rubber adhesives or two-sided adhesive tape. Preferably, the pressure-sensitive adhesive is adhesive number D-39964-B available from Fuller Co. (Germany). As a non-limiting example, pressure sensitive adhesive strips, swirls, or waves may be applied to help maintain the absorbent article in place. As used herein, the term pressure-sensitive adhesive refers to any releasable adhesive, or releasable tenacious means.

As is customary in the art, a paper release strip, which has been coated on one side, is applied to protect adhesive that may be applied to the garment-facing side of the backsheet. The coating on the paper release strip, which may be silicone, reduces the adherency to the adhesive of the coated side of the release strip. The release strip can be formed from any suitable sheet-like material which, when coated, adheres with sufficient tenacity to the adhesive to remain in place prior to use but which can be readily removed when the absorbent article is to be used.

Wings

Wings, also called, among other things, flaps, or tabs, may also be part of the absorbent article of the present disclosure. Wings and their use in sanitary protection articles are described in U.S. Pat. No. 4,687,478 to Van Tilburg; U.S. Pat. No. 4,589,876 also to Van Tilburg, U.S. Pat. No. 4,900,320 to McCoy, and U.S. Pat. No. 4,608,047 to Mattingly. The disclosures of these patents are incorporated herein by reference in their entirety. As disclosed in the above documents, wings are generally speaking flexible and configured to be folded over the edges of the underwear so that the wings are disposed between the edges of the underwear.

The overall dimensions of the disposable absorbent article of the present disclosure are preferably as follows: length is preferably in the range of about 3 inches (7.63 cm) to about 9 inches (22.9 cm). The maximum width of the anterior portion (or also known as the inner MFDL layer) is preferably in the range of about 1.5 inches (3.81 cm) to 3 inches (7.6 cm).

The absorbent article of the present disclosure may be used with conventional underwear or may be shaped to conform to thong garments. As used herein, the term thong garment includes, but is not limited to, thong underwear, thong swimming suit bottom, G-strings, Rio cut underwear, Rio-cut swimming suit bottom, Brazilian cut underwear, Brazilian cut swimming suit bottom, and any other garment that exposes the buttocks, having a narrow strip of fabric or a cord that passes between the thighs supported by a waistband, a waist cord, belt or the garment itself.

Other embodiments are also included within the scope of the present invention. For example, inert pigments may be used to produce a colored absorbent article. An example may be light or dark pigment, such as carbon black, blue, green, pink, red, etc. Additionally, patterns including multiple colors and stripes, such as tiger, zebra stripes may be included.

Embossing an Absorbent Article

The present disclosure further discloses embossing a disposable absorbent article and methods to enhance the effectiveness of embossing for stain reduction. Such an embossed absorbent article of the present invention is undertaken by applying heat and pressure to various hydrophobic barrier formulations that are applied onto an absorbent article thereby forming a permanent hydrophobic barrier that controls and directs fluid flow about an absorbent article.

In general, the embossing methods employed by this invention may be done by any embossing tool known in the art.

Embossing is typically done by feeding or placing material between the embossing tool and a back-up plate or roller, wherein the embossing tool imparts deformation onto selected portions of the material due to the shape of the embossing tool. The deformation of the material results from not only the shape of the embossing tool, but the stress put on the material by the embossing tool. The embossing tool may be made of metal, polymers, rubber, and any material having greater rigidity as compared to the material being deformed.

One preferred example of the emboss roller usable herein includes a metal roller made of metal such as iron, stainless steel or ahoy, and the like. One preferred example of the presser roller includes a metal roller or a rubber roller such as silicone or urethane or the like or a cotton roller and the like.

The emboss processing is preferably performed by means of heat embossing, namely, by heating the emboss roller. The conditions for heat embossing are preferably as follows.

The temperature of the emboss roller is preferably from 80° C. to 180° C., but is dependent on the applicable substrates used so the temperature may be lower or higher than the prescribed range. The pressure to be applied to the absorbent article precursor by the emboss roller and the presser roller is preferably from about 1.0 kgf/cm2 to about 7.5 kgf/cm2.

As disclosed herein, embossing is done with heat. For example, if the material requires heat to set the embossing pattern such that the material does not regain its original surface smoothness, heat may be employed to bond the embossed pattern.

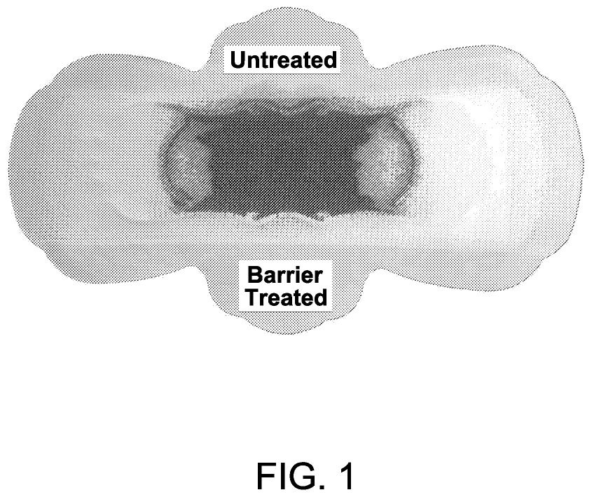

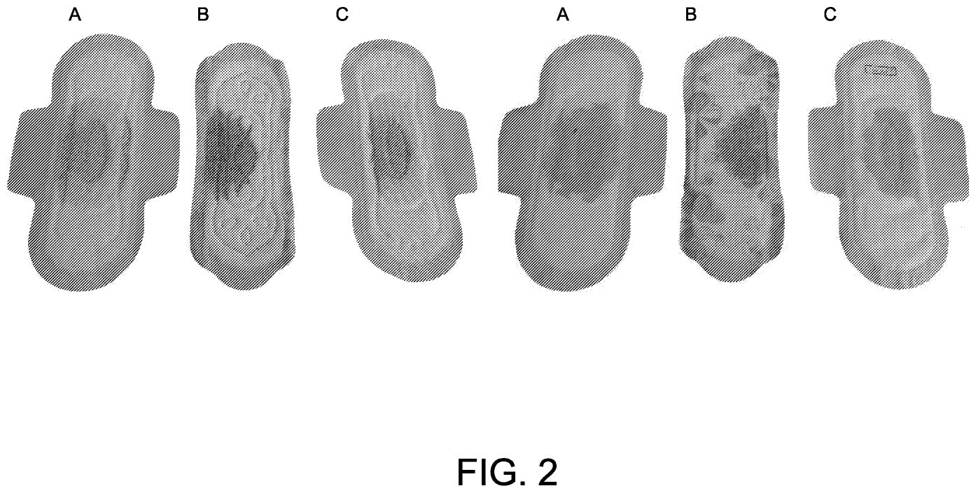

Turning to the Figures, FIGS. 1-2 depict pictures of disposable absorbent articles where one side is treated with embossing patterns applied onto a barrier treated area versus an untreated side. FIG. 1 shows the side leakage barrier made by applying a treatment barrier formulation of 60% petrolatum and 40% ozokarite wax and embossing it onto the absorbent article with heat and pressure whereby a barrier is formed from the top of the topsheet through the absorbent core and onto the backsheet. The barrier thus formed presents a 100% leakage barrier.

FIG. 2 shows an absorbent article A that is not treated with a barrier formulation, B is treated with a barrier formulation on one side of the absorbent article and C treated with a barrier formulation on both sides of the absorbent article. The barrier formulations contain 60% petrolatum and 40% ozokarite wax for absorbent article B and for absorbent article C the barrier formulation contains 60% petrolatum and 40% stearyl alcohol. Thereafter embossment of the treated area with heat and pressure takes place. Picture C show a 100% leakage barrier on both sides of the absorbent articles while picture B shows a 100% leakage barrier on one side of the absorbent article. No matter how small the absorbent article, the application of 60% petrolatum with 40% ozokarite wax or 40% stearyl alcohol on an absorbent article and embossment of the treated area with heat and pressure still resulted in a 100% leakage barrier on the treated sides of the absorbent articles. Absorbent article B is a 0.06 g/absorbent article.

Embossed patterns may be raised particularly on the top of the top sheet or absorbent core. Embossed patterns may be any shape, e.g, cone, cube, rectangle, rounded, e.g., oval, and the like.

The embossed pattern may be a continuous pattern, such as, a line; or a discontinuous (interrupted) pattern, such as, geometric shapes, e.g., rectangles, diamonds, squares, circles; letters; numbers; images; or combinations thereof.

In one embodiment of the disclosure, the pattern may be embossed onto the body facing topsheet surface of a disposable absorbent article. In yet in another embodiment of the disclosure, the pattern may be embossed onto the absorbent core of a disposable absorbent article. Such articles include, pantiliners, sanitary napkins, interlabial devices, adult incontinence devices, bandages, wipes, and diapers. Multiple patterns may be embossed onto the topsheet surface. Such multiple patterns may include, for example, a series of hatch marks, criss-crosses, bars, knurled, waves, spirals, swirls, letters, numbers, logos, pictures, and the like. Other patterns are possible and known in the art.

Formulation

Barrier formulations generally include basic ingredients, waxes, and optionally a rheology modifier. For example, the barrier formulation may include from about 5 to about 100 weight percent of base ingredients, from about 0 to about 95 weight percent of a wax, and from about 0 to about 25 weight percent of a rheology modifier based on a total weight of barrier formulations formulation. (Tables 1 and 2). The barrier formulation may include other ingredients such as a light stabilizer or an antioxidant.

TABLE 1
Formulation break down: combinations of base ingredients and waxes

| Trade name | INCI name | AA | BB | CC | DD | EE |
|---|---|---|---|---|---|---|
| Ozokerite Wax 1020P | Ozokerite Wax | 40.00 | | | | |
| Bees Wax White BP | Bees Wax | | | 3.65 | 32.28 | 16.14 |
| Snow White EC | Petrolatum | 60.00 | 60.00 | | | |
| Lanette 18 | Stearyl Alcohol | | 40.00 | 18.18 | 19.36 | |
| Lanette 22 | Behenyl Alcohol | | | 18.18 | 19.36 | |
| Lanette O | Cetearyl Alcohol | | | | | 54.86 |
| Cutina CP | Cetyl Palmitate | | | 26.67 | 28.40 | 28.40 |
| Monomuls 90 O 18 | Glyceryl Oleate | | | 2.42 | | |
| Myritol 331 | Cocoglyceride | | | 30.30 | | |
| Uvinul MC 80 | Ethylhexyl | | | 0.50 | 0.50 | 0.50 |
| | Methoxycinnamate | | | | | |
| BHT | Dibutylhydroxytoluene | | | 0.10 | 0.10 | 0.10 |

| | | | | | |
|---|---|---|---|---|---|
| Total: | 100.00 | 100.00 | 100.00 | 100.00 | 100.00 |

TABLE 2
Formulation break down: rheology modifier

| Trade name | INCI name | SOFT 50 | SOFT 50-1 | SOFT 50-3 | SOFT 50-5 |
|---|---|---|---|---|---|
| Bees Wax White BP | Bees wax | 2.58 | 2.58 | 2.58 | 2.58 |
| Lanette 18 | Stearyl Alcohol | 12.93 | 12.93 | 12.93 | 12.93 |
| Lanette 22 | Behenyl Alcohol | 12.93 | 12.93 | 12.93 | 12.93 |
| Cutina CP | Cetyl Palmitate | 19.01 | 19.01 | 19.01 | 19.01 |
| Monomuls 90 O 18 | Glyceryl Oleate | 2.00 | 2.00 | 2.00 | 2.00 |
| Myritol 318 | Caprylic/Capric Triglyceride | 50.00 | 49.00 | 47.00 | 45.00 |
| Uvinul MC 80 | Ethylhexyl Methoxycinnamate | 0.50 | 0.50 | 0.50 | 0.50 |
| Tinogard TT | Pentaerythrityl Tetra-di-butyl | 0.05 | 0.05 | 0.05 | 0.05 |
| | Hydroxyhydrocinnamate | | | | |
| Performacol 350 | C20-40 Alcohols | | 1.00 | 3.00 | 5.00 |

| | | | | |
|---|---|---|---|---|
| Total: | 100.00 | 100.00 | 100.00 | 100.00 |

Suitable waxes include, but are not limited to, paraffins, Fischer-Tropsch, microcrystalline waxes, and combinations thereof. Suitable microcrystalline waxes include, but are not limited to, BE SQUARE 175 microwax, available from Bareco Division, Petrolite Corporation, and M-5165 from Moore Munger, Shelton, Conn. Suitable polyethylene waxes include, but are not limited to, H-101 from Exxon Chemical, Houston, Tex. Suitable Fischer-Tropsch waxes include, but are not limited to, Paraflint Wax from Schumann Sasol, Hamburg, Germany. Other suitable waxes are animal wax, vegetable wax, mineral wax, silicone wax, natural or synthetic wax, vbayberry wax, bees wax, C30 alkyl dimethicone, candelila wax, carnauba wax, ceresin wax, cetyl esters, esparto, hydrogenated cotton seed oil, hydrogenated jojoba oil, hydrogenated jojoba wax, hydrogenated microcrystalline wax, hydrogenated rice bran wax, japan wax, jojoba wax, jojoba butter, jojoba esters, lanolin wax, microcrystalline wax, mink wax, motan acid wax, motan wax, ouricury wax, ozokerite wax, paraffin wax, PEG-6 beeswax, PEG-8 beeswax, rice bran wax, shellac wax, spent grain wax, spermaceti wax, stearyl dimethicone, synthetic beeswax, synthetic candelilla wax, synthetic jojoba wax or mixtures thereof.

Wax material combinations other than petrolatum and stearyl alcohol were reviewed and applied in the same percent as those combinations set forth in column BB of Table 1. Table 3 shows the summary of these results.

In order to obtain table 3, physical property tests of alternative petrolatum ingredients and barrier formulations were evaluated. A rheometer was used which provided hardness of alternative petrolatum ingredients with a teflon probe and needle penetration values with a modified needle. Specific gravity of barrier formulations were measured. Please see table 3 for these measurements.

The specific gravity of each formulation was measured with water and a 40 ml sample bottle. Specific gravity is a dimensionless number. It's the ratio of the density or mass of substance (formulation) to the density or mass of reference (water). The specific gravity was calculated as follows:
1. Prepare clean sample bottle and weigh the bottle with cap (A)
2. Put the bottle and cap in water and screw on a cap under water
3. Dry water on outside of bottle and measure weight (B)
4. Spill water and dry bottle
5. Pour hot formulation into bottle and measure the weight (C)
6. Solidify formulation and put it in the water
7. Screw on a cap under water and dry the bottle
8. Measure the weight (D)
9. Calculate specific gravity

$$Specific Graivity = \frac{amount of formulation}{amount of water} = \frac{C}{B + C - D}$$

$$( amount of water = ( B - A ) - ( D - A - C ) = B + C - D )$$

In view of the physical tests conducted, a 60% castrolatum and 40% cetearyl alcohol combination was found to have the same effectiveness barrier formulation as that of the 60% petrolatum and 40% stearyl alcohol combination. Two other combinations that were found to have the same effectiveness barrier formulations as the 60% petrolatum and 40% stearyl alcohol are 60% Sebapet L and 40% stearyl alcohol and 60% Sonnenatural J-207 and 40% stearyl alcohol.

Overall, the hydrophobic barrier formulations of petrolatum and ozokerite wax or stearyl alcohol, castrolatum and 40% cetearyl alcohol, sebapet L and stearyl alcohol and sonnenatural J-207 and stearyl alcohol showed leading barrier function alone, however, it has been found that the combination of the barrier treatment and embossing delivered much more advantageous leakage protection capability. (See FIGS. 1 and 2 and table 3). When the embossing patterns were applied on barrier-treated areas, the molten barrier by heat penetrated into layers and built 100% hydrophobic barriers through all the layers.

TABLE 3
Summary of material and barrier properties

| | Material | | | | | Barrier with Stearyl Alcohol/Cetearyl Alcohol | | | |
|---|---|---|---|---|---|---|---|---|---|
| | | | Cost | Fluo- | Hardness | Fluo- | | Penetration | Specific |
| Trade Name | Supplier | Use Level* | (USD/kg) | rescence | (g/cm²) | rescence | Compatibility | (mm) | Gravity |
| AKOGEL | AAK | 5-15% | | Weak | 108,000.00 | Fluo- | Acceptable | 2.96 | 0.904 |
| | | | | | | rescent | | | |
| AKOGEL PLUS | AAK | 5-15% | | Weak | 14,000.00 | None | Acceptable | 2.99 | 0.914 |
| CASTROLATUM | VERTELLUS | 70-100% | 10.00 | None | 3,180.00 | None | Acceptable** | 4.45 | 0.943 |
| CREMERLIN | IOI | | 18.00 | None | 97.17 | None | Acceptable | 5.94 | 0.911 |
| PURA | | | | | | | | | |
| CRODANAL | CRODA | 1-25% | | None | 20,300.00 | Fluo- | Acceptable | 3.99 | 0.908 |
| SWL-SO | | | | | | rescent | | | |
| LIPO-HB | WNC | 1-98% | 28.00 | None | Overloaded | None | Acceptable | 4.39 | 0.935 |
| LIPO-HBE | WNC | 1-3% | | None | 49.47 | Fluo- | Acceptable | 4.12 | 1.000 |
| | | | | | | rescent | | | |
| LIPO-HF | WNC | 1-80% | | None | Overloaded | None | Fail | 3.92 | 0.935 |
| MEDIANL-SL | CRODA | 1-10% | | None | 27,800.00 | Fluo- | Acceptable | 2.43 | 0.963 |
| | | | | | | rescent | | | |
| SEBAPET L | IGI Wax | | 11.00 | None | 3,750.00 | None | Acceptable | 4.48 | 0.923 |
| SOFTISAN 649 | IOI | | | None | 46,033.33 | Fluo- | Acceptable | 2.46 | 0.979 |
| | | | | | | rescent | | | |
| SONNENATURAL | SONNEBORN | | 9.00 | None | 320.33 | None | Acceptable | 2.30 | 0.913 |
| J-207 | | | | | | | | | |
| VERSAGEL ME | PENRENCO | | | None | 88.23 | None | Fail | 9.68 | 0.845 |
| 16000 | | | | | | | | | |

*Recommended use level by manufacturers
**CASTROLATUM was not compatible with Stearyl Alcohol, but it showed good stability with Cetearyl Alcohol.

The specification and examples above are presented to aid in the complete and non-limiting understanding of the invention disclosed herein. Since many variations and embodiments of the invention can be made without departing from its spirit and scope, the invention resides in the claims hereinafter appended.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

Those skilled in the art will recognize that the present disclosure may be manifested in a variety of forms other than the specific embodiments described and contemplated herein. Specifically, the various features described with respect to the various embodiments and figures should not be construed to be applicable to only those embodiments and/or figures. Rather, each described feature may be combined with any other feature in various contemplated embodiments, either with or without any of the other features described in conjunction with those features. Accordingly, departure in form and detail may be made without departing from the scope of the present disclosure as described in the appended claims.

What is claimed is:

1. A disposable absorbent article comprising:
a) an outer cover;
b) a liquid permeable topsheet which is connected in superposed relation to the outer cover;
c) an absorbent core which is located between the topsheet and a liquid impermeable backsheet;
d) a barrier formulation on at least a portion of the topsheet surface which includes from about 5 to about 100 weight percent of a base ingredient, from about 0 to about 95 weight percent of a wax, and from about 0 to about 25 weight percent of a rheology modifier; and
e) an embossment pattern wherein the embossment pattern is applied onto the barrier treated area whereby forming a barrier along the full length of the embossment.

2. The disposable absorbent article according to claim 1, wherein the disposable absorbent article may be pantiliners, sanitary napkins, interlabial devices, adult incontinence devices, bandages, wipes, diapers and the like.

3. The absorbent article according to claim 1 wherein the barrier formulation is a combination of base ingredients and waxes.

4. The absorbent article according to claim 3, wherein the barrier formulation comprises about 35-45% ozokerite wax and about 55% to about 65% petrolatum; or about 35-45% stearyl alcohol and about 55% to about 65% petrolatum; or about 55%-65% castrolatum and from about 35-45% cetearyl alcohol; or about 55-65% Sebapet L and from about 35-45% stearyl alcohol; or
about 55-65% Sonnenatural J-207 and from about 35-45% stearyl alcohol.

5. The absorbent article according to claim 1 wherein the embossed pattern may be a continuous pattern such as a line or may be in a discontinuous pattern such as geometric shapes like rectangles, diamonds, squares, circles, letters, numbers, images, or combinations thereof.

6. The disposable absorbent article according to claim 1, wherein the embossing pattern thickness is at least 15% of the total thickness of the absorbent article.

7. The disposable absorbent article according to claim 1, wherein the embossing pattern has a width from about 9 mm to about 17 mm and the length of the pattern is from about 70 mm to about 100 mm.

8. A process for making a barrier in a disposable absorbent article comprising:
applying a barrier formulation onto the top surface of a body facing topsheet;
embossing the barrier formulation with heat and pressure;
whereby forming a barrier along the full length of the embossed area thus creating a barrier that extends from the top sheet through an absorbent core to a back sheet.

9. The process according to claim 8 wherein the barrier formulation is a combination of base ingredients and waxes.

10. The process according to claim 9, wherein the barrier formulation comprises about 35-45% ozokerite wax and about 55% to about 65% petrolatum; or about 35-45% stearyl alcohol and about 55% to about 65% petrolatum; or about 55%-65% castrolatum and from about 35-45% cetearyl alcohol; or about 55-65% Sebapet L and from about 35-45% stearyl alcohol; or about 55-65% Sonnenatural J-207 and from about 35-45% stearyl alcohol.

11. The process according to claim 8 wherein the barrier formulation is applied to the top sheet with a pressure from about 1.0 kgf/cm2 to about 7.5 kgf/cm2.

12. The process according to claim 8 wherein the barrier formulation is applied to the absorbent core with a pressure from about 1.0 kgf/cm2 to about 7.5 kgf/cm2.

13. A process for making a barrier in a disposable absorbent article comprising:
applying a barrier formulation onto an absorbent core;
embossing the barrier formulation with heat and pressure;
whereby forming a barrier along the full length of the embossed area thus creating a barrier that extends from the absorbent core to a back sheet.

14. The process according to claim 13, wherein the embossing pattern thickness is at least 15% of the total thickness of the absorbent article.

15. The process according to claim 13, wherein the embossing pattern has a width from about 9 mm to about 17 mm and the length of the pattern is from about 70 mm to about 100 mm.

16. The process according to claim 13, wherein the disposable absorbent article may be pantiliners, sanitary napkins, interlabial devices, adult incontinence devices, bandages, wipes, diapers and the like.

17. The process according to claim 13, wherein the barrier formulation is a combination of base ingredients and waxes.

18. The process according to claim 17, wherein the barrier formulation comprises about 35-45% ozokerite wax and about 55% to about 65% petrolatum;
or about 35-45% stearyl alcohol and about 55% to about 65% petrolatum; or
or about 55%-65% castrolatum and from about 35-45% cetearyl alcohol; or about 55-65% Sebapet L and from about 35-45% stearyl alcohol; or about 55-65% Sonnenatural J-207 and from about 35-45% stearyl alcohol.

19. The process according to claim 12, wherein the barrier formulation is applied to the top sheet with a pressure from about 1.0 kgf/cm2 to about 7.5 kgf/cm2.

20. The process according to claim 13, wherein the barrier formulation is applied to the absorbent core with a pressure about 1.0 kgf/cm2 to about 7.5 kgf/cm2.
